(19) United States
(12) Patent Application Publication
(10) Pub. No.: US 2026/0072296 A1
(43) Pub. Date: Mar. 12, 2026

(54) OPTICALLY DYNAMIC HEAD-MOUNTED DISPLAY DEVICE (HMDD)

(71) Applicant: Charter Communications Operating, LLC, St. Louis, MO (US)

(72) Inventors: Hany Heikal, Aurora, CO (US); Mohamed Daoud, Aurora, CO (US)

(21) Appl. No.: 18/883,165

(22) Filed: Sep. 12, 2024

(51) Int. Cl. G02C 7/08; A61B 3/028; G06F 3/04847

(52) U.S. Cl. CPC G02C 7/08; A61B 3/028; G06F 3/04847

(57) ABSTRACT

A head-mounted display device (HMDD) operable to provide extended-reality (XR) services is provided. The HMDD includes a display, adjustable lenses that include an active medium proximate the display, a memory, and a processor device coupled to the memory. The processor device is operable to determine a visual acuity metric associated with a user and, based on the visual acuity metric associated with the user, adjust an optical characteristic of the adjustable lenses, such as refractive index, lens curvature, and/or the like. In some examples, the adjustable lenses are electro-optical lenses comprising an active liquid crystal medium that is operable to adjust an optical characteristic associated therewith in response to applied electrical stimuli. In some examples, the adjustable lenses are opto-mechanical lenses comprising an active fluid medium within a flexible membrane that is operable to adjust an optical characteristic associated therewith in response to applied mechanical stimuli.

### BACKGROUND

Virtual reality (VR) environments and/or augmented reality (AR) environments enable a user to experience an immersive virtual world using a head-mounted display device (HMDD) that renders high-resolution VR content and/or AR content. The VR environment and/or the AR environment may be generated by a network computing device and streamed to a user’s HMDD and/or may be generated by a client computing device of the user based on downloaded VR content and/or AR content. In some examples, HMDDs may be used to watch streaming video content (e.g., sporting events, moving, etc.) within the VR environment and/or the AR environment.

### SUMMARY

The examples disclosed herein provide a head-mounted display device (HMDD) operable to dynamically configure and/or adjust the optical characteristics associated with its display device to accommodate the vision needs of users based on a visual acuity metric associated with the user.

In one implementation, a method is provided. The method includes determining, by a head-mounted display device (HMDD) comprising adjustable lenses, a visual acuity metric associated with a user of the HMDD. The method further includes adjusting, by the HMDD, an optical characteristic of the adjustable lenses based on the visual acuity metric associated with the user.

In another implementation, a head-mounted display device (HMDD) is provided. The HMDD includes a display. The HMDD further includes adjustable lenses proximate the display. The adjustable lenses include an active medium. The HMDD further includes a memory and a processor device coupled to the memory. The processor device is operable to determine a visual acuity metric associated with a user of the HMDD and adjust an optical characteristic of the adjustable lenses based on the visual acuity metric associated with the user.

In another implementation, a non-transitory computer-readable medium is provided. The non-transitory computer-readable medium includes executable instructions configured to cause a processor device to determine a visual acuity metric associated with a user of a head-mounted display device (HMDD) comprising adjustable lenses adjust an optical characteristic of the adjustable lenses based on the visual acuity metric associated with the user.

Individuals will appreciate the scope of the disclosure and realize additional aspects thereof after reading the following detailed description of the examples in association with the accompanying drawing figures.

### BRIEF DESCRIPTION OF THE DRAWINGS

The accompanying drawing figures incorporated in and forming a part of this specification illustrate several aspects of the disclosure and, together with the description, serve to explain the principles of the disclosure.

FIG. 1 is a block diagram suitable for performing dynamic and adaptive vision correction in a head-mounted display device (HMDD) according to some implementations;

FIGS. 2A-2D are sequence diagrams illustrating messages communicated between and actions taken by certain components illustrated in FIG. 1 to perform dynamic and adaptive vision correction in a head-mounted display device (HMDD) according to some implementations;

FIG. 3 is a flowchart of a method for dynamically configuring adjustable lenses of a head-mounted display device (HMDD) according to some implementations;

Figure 4 is a block diagram of a computing device suitable for implementing examples disclosed herein; and

FIG. 5 is a block diagram of a head-mounted display device (HMDD) suitable for implementing examples disclosed herein.

### DETAILED DESCRIPTION

The examples set forth below represent the information to enable individuals to practice the examples and illustrate the best mode of practicing the examples. Upon reading the following description in light of the accompanying drawing figures, individuals will understand the concepts of the disclosure and will recognize applications of these concepts not particularly addressed herein. It should be understood that these concepts and applications fall within the scope of the disclosure and the accompanying claims.

Any flowcharts discussed herein are necessarily discussed in some sequence for purposes of illustration, but unless otherwise explicitly indicated, the examples and claims are not limited to any particular sequence or order of steps. The use herein of ordinals in conjunction with an element is solely for distinguishing what might otherwise be similar or identical labels, such as “first message” and “second message,” and does not imply an initial occurrence, a quantity, a priority, a type, an importance, or other attribute, unless otherwise stated herein. The term “about” used herein in conjunction with a numeric value means any value that is within a range of ten percent greater than or ten percent less than the numeric value. As used herein and in the claims, the articles “a” and “an” in reference to an element refers to “one or more” of the element unless otherwise explicitly specified. The word “or” as used herein and in the claims is inclusive unless contextually impossible. As an example, the recitation of A or B means A, or B, or both A and B. The word “data” may be used herein in the singular or plural depending on the context. The use of “and/or” between a phrase A and a phrase B, such as “A and/or B” means A alone, B alone, or A and B together.

Virtual reality (VR) environments and/or augmented reality (AR) environments enable a user to experience an immersive virtual world using a head-mounted display device (HMDD) that renders high-resolution VR content and/or AR content. The VR environment and/or the AR environment may be generated by a network computing device and streamed to a user’s HMDD and/or may be generated by a client computing device of the user based on downloaded VR content and/or AR content. In some examples, HMDDs may be used to watch streaming video content (e.g., sporting events, moving, etc.) within the VR environment and/or the AR environment.

HMDDs that provide VR and AR functionality may include a display device (e.g., a screen), an input device (e.g., a digital crown, buttons, etc.), appropriate sensors for detecting position and movement of the HMDD (e.g., accelerometers, compasses, positioning systems, gyroscopes, etc.), and appropriate software. In this way, HMDDs are operable to provide an immersive experience allowing a user to perceive the VR and/or AR environment (hereinafter “virtual environment”) in which virtual elements are integrated into or overlaid onto the real-world environment. In some examples, the entire virtual environment perceived by the user may be computer generated.

However, conventional HMDDs pose significant challenges for users with vision impairments. For instance, visually impaired users must typically purchase additional prescription lenses (e.g., prescription inserts) specially designed to be used with a HMDD in order to experience the aforementioned virtual environments. Like prescription lenses for eyeglasses, obtaining prescription inserts for HMDDs is a labor-intensive and expensive process for a user.

Accordingly, example aspects of the present disclosure are directed to an HMDD having adjustable lenses integrated therein. As used herein, adjustable lenses are “integrated” with the HMDD when the adjustable lenses are electrically and/or mechanically coupled to the HMDD. For instance, in some examples, HMDDs of the present disclosure may include adjustable lenses that form part of the HMDD itself. Additionally and/or alternatively, in some examples, HMDDs of the present disclosure may include adjustable lenses that are attachable and detachable from a housing of the HMDD.

As will be discussed in greater detail below, the adjustable lenses of the present disclosure include an active medium that is operable to adjust one or more optical characteristics (e.g., refractive index, lens curvature, etc.) of the HMDD in response to external stimuli being applied thereto. As one example, the adjustable lenses may be electro-optical lenses. In particular, electro-optical lenses include an active liquid crystal medium which, when an external stimulus (e.g., electrical stimuli) is applied thereto (e.g., by a controller of the HMDD), adjusts (e.g., changes) one or more optical characteristics of the electro-optical lenses. It should be understood that, as used herein, an “active liquid crystal medium” refers to an active medium that includes liquid crystals. Additionally and/or alternatively, as another example, the adjustable lenses may be opto-mechanical lenses. In particular, opto-mechanical lenses include an active fluid medium within a flexible membrane which, when an external stimulus (e.g., mechanical stimuli) is applied thereto (e.g., by a controller of the HMDD), adjusts (e.g., changes) one or more optical characteristics of the opto-mechanical lenses. It should be understood that, as used herein, an “active fluid medium within a flexible membrane” refers to an active medium that includes a fluid within a flexible membrane. In this way, an HMDD of the present disclosure is operable to provide dynamic and adaptive vision correction to the user.

The adjustable lenses of the HMDD may be configured and/or adjusted based on a visual acuity metric associated with the user. As will be discussed in greater detail below, the visual acuity metric associated with the user may characterize and/or otherwise be associated with a quality and/or a clarity of the user’s vision.

In some examples, the HMDD may determine the visual acuity metric associated with the user by providing a user-selectable control to the user (e.g., via a display device). As one non-limiting illustrative example, the user-selectable control may be an interactive user interface (UI) slider, such as a virtual slider implemented as a software function, that includes a range of different vision settings for the adjustable lenses. For instance, the range of different vision settings may include a farsightedness vision setting, a plurality of intermediate vision settings, and a nearsightedness vision setting. In particular, in the farsightedness vision setting, the adjustable lenses may be configured to compensate for hyperopia refractive error (e.g., farsightedness). Likewise, in the nearsightedness vision setting, the adjustable lenses may be configured to compensate for myopia refractive error (e.g., nearsightedness). Furthermore, at least one of the plurality of intermediate vision settings may correspond to a neutral vision setting in which the adjustable lenses are configured to compensate for no refractive error. The user may scroll through the various different vision settings in the range and, via a user input device, select one of the vision settings as the selected vision setting for the adjustable lenses. In response, the HMDD may determine the visual acuity metric for the user and, subsequently, configure and/or adjust the adjustable lenses based on the visual acuity metric (e.g., by providing external stimuli to the adjustable lenses’ active medium to adjust the one or more optical characteristics associated therewith).

In some examples, the HMDD may determine the visual acuity metric associated with the user by obtaining biometric data associated with the user. As will be discussed in greater detail below, the biometric data may be obtained by one or more sensors of the HMDD (e.g., biometric sensors, refractometer, etc.) and may characterize a refractive error associated with the user (e.g., the refractive error of the user’s eyes). In such examples, the HMDD may determine the visual acuity metric associated with the user based on the biometric data and, in response, may configure and/or adjust the adjustable lenses based on the visual acuity metric (e.g., by providing external stimuli to the adjustable lenses’ active medium to adjust the one or more optical characteristics associated therewith).

Subsequent to configuring and/or adjusting the optical characteristic(s) of the adjustable lenses (e.g., based on the visual acuity metric), the HMDD may fine-tune the vision setting by obtaining (e.g., receiving) user feedback data. For instance, in some examples, the HMDD may provide a user-selectable control to the user (e.g., via a display device), such as an interactive UI slider having a range of different vision settings. In some examples, the user-selectable control may include the same range of different vision settings as described above. In other examples, the user-selectable control may include a different range of different vision settings, such as a narrower range of vision settings than the range described above. The user may provide, and the HMDD may receive, user feedback data that includes a tuned vision setting for the adjustable lenses, and the HMDDD may tune (e.g., adjust) the optical characteristic(s) of the adjustable lenses based on the received tuned vision setting.

The present disclosure provides a number of technical effects and benefits, including improvements to computing technology. As one example, the present disclosure provides an HMDD that is operable to dynamically and adaptively configure and/or adjust its optical characteristics to accommodate the various vision-related needs of its users. As such, HMDDs of the present disclosure provide significantly greater accessibility to user having vision impairments relative to conventional HMDDs. In this way, the present disclosure provides cost- and time-savings to users with vision impairments by obviating the need (by the user) to spend time and/or money obtaining custom optical inserts. Moreover, by configuring and/or adjusting the adjustable lenses during the boot process, HMDDs of the present disclosure enhance and improve the overall performance of the HMDD and reduces or eliminates manual actions that would otherwise be necessary. Even further, the example HMDDs of the present disclosure continuously adapt to the vision needs of the user by determining a visual acuity metric associated with the user, thereby providing a continuous, dynamic, clean, and personalized viewing experience for the user.

Additionally, example aspects of the present disclosure also provide resulting improvements to computing technology tasked with providing AR and/or VR serves to users, such as the HMDD itself, client devices coupled to the HMDD (e.g., smartphone, laptop, tablet device, etc.), network computing devices (e.g., server computing devices) implemented by a service provider, and/or the like. As one example, by dynamically adapting to the various vision-related needs of its users, unnecessary computational operations by the HMDD and/or other associated computing devices may be reduced, such as iterative and/or repetitive operations taken by the HMDD in response to the user input mistakes attributable to vision-related impairments. Likewise, processing and storage requirements for the HMDD and/or other associated computing devices and systems may be directly reduced, ultimately resulting in more efficient resource use on both the user-side and the service provider-side. As one example, by reducing the number of user input-related errors and/or other vision-related accessibility features (e.g., text-to-speech, increased UI size(s), etc.), the HMDD may reduce its power draw and its processing requirements, which directly improves operation speeds for the HMDD and other associated computing devices (e.g., client computing device(s), server computing system(s), etc.). In this way, valuable computing resources within the HMDD and/or other associated computing devise and systems may be reserved for other tasks, such as obtaining and rendering AR and/or VR environments, streaming video content, and/or the like.

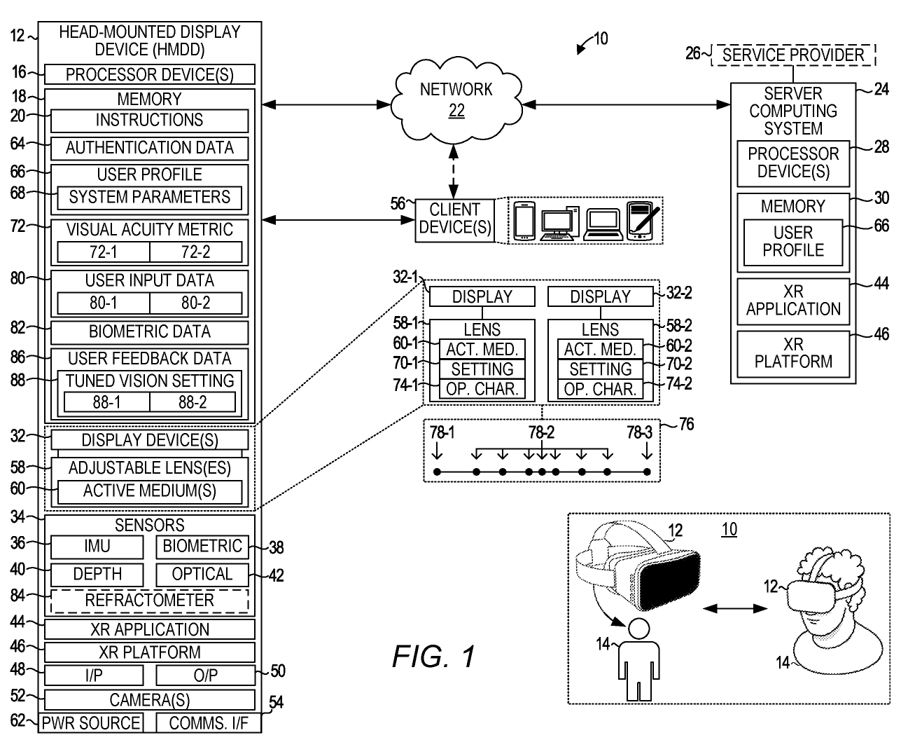

FIG. 1 is an example block diagram of an environment 10 suitable for implementing dynamic and adaptive vision correction in a display device operable to provide extended reality (XR) services. It should be understood that, as used herein, “extended reality (XR)” is a generalized term that refers to and encompasses a wide variety of immersive technologies, such as “augmented reality (AR),” “virtual reality (VR),” “mixed reality (MR),” and/or the like. Put differently, “extended reality (XR)” environments refer to an entire spectrum of immersive virtual environments, ranging from fully virtual (e.g., computer-generated) environments (e.g., VR environments) to partially virtual environments having computer-generated digital information and/or virtual objects overlaid onto a non-virtual real-world environment (e.g., AR environments), as well as virtual environments that merge (and allow for interactions between) digital and real-word elements (e.g., MR environments).

As shown in FIG. 1, the environment 10 includes a head-mounted display device (HMDD) 12, which is operable to provide XR serves to a user 14. The HMDD 12 may be worn, for instance, on a head of the user 14 to view virtual content (e.g., virtual reality (VR) content, augmented reality (AR) content, mixed reality (MR) content, etc.) on the HMDD 12.

For instance, in some examples, the HMDD 12 may be a VR computing device that is operable to provide VR functionality to the user 14. In such examples, the HMDD 12 fully immerses the user 14 in completely digital virtual environment (e.g., a VR environment) that replaces the non-virtual real-world environment (e.g., physical surroundings) of the user 14. In other examples, the HMDD 12 may be an AR computing device that is operable to provide AR functionality to the user 14. In such examples, the HMDD 12 overlays (e.g., superimposes) computer-generated digital information and/or virtual objects onto a digital representation (e.g., an AR environment) of the non-virtual real-world environment (e.g., physical surroundings) of the user 14. In other examples, the HMDD 12 may be an MR computing device that is operable to provide MR functionality to the user 14. In such examples, the HMDD 12 merges digital information and/or objects with the non-digital real-world objects in the physical surroundings of the user 14 to generate a MR environment. While similar to AR environments, MR environments allow for more complex interactions between the user 14 and the MR environment.

Those having ordinary skill the art, using the disclosures provided herein, will understand that the HMDD 12 may be any suitable computing device operable to provide XR functionality (e.g., AR functionality, VR functionality, MR functionality, etc.), such as XR goggles, XR glasses, optical see-through head-mounted devices, video see-through head-mounted devices, and/or the like.

The HMDD 12 may include a processor device 16. The processor device 16 may include any computing or electronic device(s) capable of executing software instructions to implement the functionality described herein. For example, the processor device 16 may be one or more of a processor, processor cores, a controller and an arithmetic logic unit, a central processing unit (CPU), a graphics processing unit (GPU), a digital signal processor (DSP), an image processor, a microcomputer, a field programmable array, a programmable logic unit, an application-specific integrated circuit (ASIC), a microprocessor, a microcontroller, etc., and combinations thereof, including any other device capable of responding to and executing instructions in a defined manner. The processor device 16 may be a single processor device and/or a plurality of processor devices that are operatively connected, for instance, in a parallel configuration.

The HMDD 12 may further include a memory 18. The memory 18 may be communicatively coupled to the processor device 16. The memory 18 may include executable instructions 20 that, when executed, cause the processor device 16 to perform operations, such as any of the operations described herein. In some examples, the memory 18 includes a controller (not shown) operable to implement the functionality described herein. Because the controller (not shown) is a component of the HMDD 12, functionality implemented by the controller (not shown) may be attributed to the HMDD 12 generally. Moreover, in examples where the controller (not shown) comprises software instructions (e.g., instructions 20) that program the processor device 16 to carry out the functionality described herein, functionality implemented by the controller (not shown) may be attributed to the processor device 16 and/or to the HMDD 12 generally.

The memory 18 may be or otherwise include any device(s) capable of storing data, including, but not limited to, volatile memory (random access memory, etc.), non-volatile memory, storage device(s) (e.g., hard drive(s), solid state drive(s), etc.). For example, the memory device 18 may include one or more non-transitory computer-readable storage mediums, such as such as a Read Only Memory (ROM), Programmable Read Only Memory (PROM), Erasable Programmable Read Only Memory (EPROM), and flash memory, a USB drive, a volatile memory device such as a Random Access Memory (RAM), an internal or external hard disk drive (HDD), floppy disks, a blue-ray disk, or optical media such as CD ROM discs and DVDs, and combinations thereof. However, examples of the memory device 18 are not limited to the above description, and the memory device 18 may be realized by other various devices and structures as would be understood by those having ordinary skill in the art.

The HMDD 12 may communicate with one or more computing devices and/or computing systems via a network 22. The network 22 may include one or more different transmission mediums, such as, by way of non-limiting example, an optical transmission medium, an electrical transmission medium, a wireless transmission medium, and/or any combination thereof. The network 22 may be any suitable communications network, such as, by way of non-limiting example, a local area network (LAN), wireless local area network (WLAN), wide area network (WAN), personal area network (PAN), virtual private network (VPN), or the like. For example, wireless communication between elements of the examples described herein may be performed via a wireless LAN, Wi-Fi, Bluetooth, ZigBee, Wi-Fi direct (WFD), ultra wideband (UWB), infrared data association (IrDA), Bluetooth low energy (BLE), near field communication (NFC), a radio frequency (RF) signal, and the like. For example, wired communication between elements of the examples described herein may be performed via a pair cable, a coaxial cable, an optical fiber cable, an Ethernet cable, and the like. Communication over the network can use a wide variety of communication protocols (e.g., TCP/IP, HTTP, SMTP, FTP), encodings or formats (e.g., HTML, XML), and/or protection schemes (e.g., VPN, secure HTTP, SSL).

In some examples, the HMDD 12 may be communicatively coupled to a server computing system 24 via the network 22, which may be owned and/or operated by a service provider 26. In the example depicted in FIG. 1, the server computing system 24 is an edge server of a large network via which the service provider 26 provides services, such as video services, data services, and/or the like, to thousands and/or millions of customers. The HMDD 12 may communicate with the server computing system 24 via one or more intermediate devices, such as a wireless gateway router (not shown), that are coupled to the network 22 via a communications medium (e.g., coaxial cable, fiber, etc.). In some examples, the HMDD 12 may be connected to a local area network (LAN) implemented by the wireless gateway router that is different than the network 22 to which the server computing system 24 is connected. In some examples, the HMDD 12 may communicate with the server computing system 24 via intermediate telecommunications equipment, such as 4G telecommunications equipment, 5G telecommunications equipment, satellite communications equipment, and/or the like.

The server computing system 24 may include a processor device 28 and a memory 30, such as any of the processor device(s) and/or memory device(s) described herein. For examples, the processor device 28 may include any computing or electronic device capable of executing software instructions to implement the functionality described herein, and the memory 30 may be or otherwise include any device(s) capable of storing data, including, but not limited to, volatile memory (random access memory, etc.), non-volatile memory, storage device(s) (e.g., hard drive(s), solid state drive(s), etc.).

As noted above, the HMDD 12 may be worn, for instance, on a head of the user 14 and may be operable to provide VR functionality and/or AR functionality to the user 14. In particular, the HMDD 12 may include a display device 32. The display device 32 may include an active display, such as a Liquid Crystal on Silicon (LCOS) display, a Light-Emitting Diode (LED) display, an Organic Light-Emitting Diode (OLED) display, a Liquid Crystal Display (LCD), an Active Matrix Organic Light-Emitting Diode (AMOLED) display, a flexible display, a 3D display, a Plasma Display Panel (PDP), a Cathode Ray Tube (CRT) display, and/or the like, on which imagery is presented. It should be understood that any suitable display device 32 may be used without deviating from the scope of the present disclosure.

The HMDD 12 may further include one or more sensors 34. For instance, in some examples, the HMDD 12 may include an inertial measurement unit sensor (IMU) 36. The IMU 36 may be operable to generate data (e.g., roll data, yaw data, pitch data, etc.), and the processor device 16 may use the data generated by the IMU 36 to determine in which direction the HMDD 12 is oriented and, thus, what real-word objects are within a field of view (FOV) of the HMDD 12. The HMDD 12 may further include one or more biometric sensors 38, such as any suitable biometric sensor operable to obtain biometric data associated with the user 14. The HMDD 12 may further include one or more depth sensors 40 (e.g., depth cameras). The depth sensors 40 may generate depth data, and the processor device 16 may use the depth data to generate a spatial map of the physical space surrounding the user 14. The HMDD 12 may further include one or more optical sensors 42 (e.g., one or more built-in interior cameras) operable to track the eyes of the user 14 when the user 14 is wearing the HMDD 12.

The HMDD 12 may further include one or more XR applications 44, such as any suitable application that allows the HMDD 12 to implement and provide (to the user 14) a computer-generated digital environment, such as an XR environment (e.g., AR environment, VR environment, MR environment, etc.). As described herein, the computer-generated digital environment (e.g., AR environment, VR environment, etc.) provided by the HMDD 12 to the user 14 includes scenes, objects, etc. that appear to be real which, in turn, makes the user 14 feel as if they are immersed in their surroundings. For instance, the one or more XR applications 44 may be related to, by way of non-limiting example, gaming, social media, medicine, vehicles, military and space applications, and/or the like. In some examples, the server computing system 24 may also include the one or more XR applications 44.

The HMDD 12 may further include one or more XR platforms 46, such as any suitable AR platform, VR platform, MR platform, and/or the like, that hosts and/or provides the XR applications 44 to the HMDD 12 (and, in some examples, to the server computing system 24). In some examples, the server computing system 24 may also include the one or more XR platforms 46.

The HMDD 12 may further include an input device 48 that is operable to receive an input (e.g., feedback) from the user 14 (e.g., via a user-selectable control). The input device 48 may be used by the user 14 to provide an input (e.g., a voice input, a touch input, a gesture input, a click via a mouse or a remote controller, etc.) to the HMDD 12 to execute any of the processes described herein. By way of non-limiting example, the input device 48 may include one or more of a keyboard (e.g., a physical keyboard, virtual keyboard, etc.), a mouse, a joystick, a button, a switch, an electronic pen or stylus, a gesture recognition sensor (e.g., to recognize gestures of a user including movements of a body part), an input sound device or voice recognition sensor (e.g., a microphone to receive a voice command), a track ball, a remote controller, a portable (e.g., a cellular or smart) phone, a dial, a digital crown, and/or the like. The input device 48 may be integrated with the HMDD 12 and/or may be communicatively coupled to the HMDD 12. For example, the user 14 may hold a remote controller having buttons, switches, a keyboard, etc., to provide a user input for executing a function of the HMDD 12, where the user input may be transmitted from the remote controller to the HMDD 12 in a wired and/or a wireless manner. The input device 48 may also be embodied by a touch-sensitive display device having, for instance, a touchscreen capability. It should be understood that any suitable input device 48 may be used without deviating from the scope of the present disclosure. In some examples, the user 14 may adjust an immersion level associated with the HMDD 12 via the input device 48. For instance, the HMDD 12 may be configured in a plurality of immersion levels, such as a VR immersion level (e.g., the user 14 immersed in a VR environment), an MR immersion level (e.g., the user 14 immersed in an MR environment), an AR immersion level (e.g., the user 14 immersed in an AR environment), and/or the like.

The HMDD 12 may further include an output device 50 that is operable to provide an output to the user 14. The output device 50 may be any suitable output device and may be operable to provide various indications, alerts, notifications, etc. to the user 14 and/or other entities in the physical surroundings of the user 14. By way of non-limiting example, the output device 50 may be one or more of an audio device (e.g., speakers), a haptic device operable to provide haptic feedback to the user 14, a light source (e.g., one or more LEDs) operable to provide visual feedback to the user 14, and/or the like. In some examples, the output device 50 may be an outward-facing display device, such as a display device facing in a similar direction as the FOV associated with the HMDD 12. It should be understood that any suitable output device 50 may be used without deviating from the scope of the present disclosure.

The HMDD 12 may further include one or more cameras 52. The one or more cameras 52 may include any suitable imaging sensor, such as, by way of non-limiting example, a complementary metal-oxide-semiconductor (CMOS), a charge-coupled device (CCD), and/or the like. In some examples, the one or more cameras 52 may be externally mounted on the HMDD 12 and may be oriented at different angles with respect to one another (e.g., a forward direction, a rearward direction, an oblique direction, etc.) so that images from the different directions can be captured by the one or more cameras 52. The one or more cameras 52 may be any suitable image-capture device operable to capture, detect, and/or recognize a behavior, figure, expression, status, etc. of the user 14 and/or the physical environment of the user 14.

The HMDD 12 may further include a communications interface 54, such as any suitable communications interface for communicating (via the network 22) with one or more computing devices, such as the server computing system 24, one or more client computing devices 56 (e.g., mobile phone, tablet device, laptop, desktop, etc.).

The HMDD 12 may further include a first adjustable lens 58-1 and a second adjustable lens 58-2 (collectively, “adjustable lens(es) 58”) proximate the display device 32. The adjustable lenses 58 may be integral with and/or insertable to the HMDD 12. The adjustable lenses 58 may be between the user 14 and the display device 32 along an optical path of the eyes of the user 14 (e.g., having an optical path directed towards the user 14). As will be discussed in greater detail below, the adjustable lenses 58 may include a first active medium 60-1 (e.g., first adjustable lens 58-1) and a second active medium 60-2 (e.g., second adjustable lens 58-2) (collectively, active medium(s) 60). In some examples, the adjustable lenses 58 may be electro-optical lenses that include an active liquid crystal medium (e.g., active medium 60) which, when an external stimulus (e.g., electrical stimuli) is applied thereto (e.g., by processor device(s) 16), adjusts (e.g., changes) one or more optical characteristics (e.g., refractive index, lens curvature, etc.) of the adjustable lenses 58. In some examples, the adjustable lenses 58 may be opto-mechanical lenses that include an active fluid medium within a flexible membrane (e.g., active medium 60) which, when an external stimulus (e.g., mechanical stimuli) is applied thereto (e.g., by processor device(s) 16), adjusts (e.g., changes) one or more optical characteristics (e.g., refractive index, lens curvature, etc.) of the adjustable lenses 58.

The HMDD 12 may further include a power source 62, such as, by way of non-limiting example, any suitable internal and/or external battery. It should be understood that, although depicted as a battery in Figure 1, the HMDD 12 may include any suitable power source 62 without deviating from the scope of the present disclosure. The power source 62 and, hence, the HMDD 12 may be activated in response to detecting a power-on trigger. For instance, in some examples, the HMDD 12 may include an input device (e.g., input device 48) that, when, activated, provides a power-on trigger to the HMDD 12 and, in response, initiates a boot process of the HMDD 12. In some examples, the HMDD 12 may include one or more sensors (e.g., sensor(s) 34) that provide a power-on trigger to the HMDD 12 in response to, by way of non-limiting example, detecting a gesture of the user 14, detecting a change in position (e.g., via IMU 36) of the HMDD 12, and/or the like. It should be understood that, as used herein, a “power-on trigger” refers to any trigger event that initiates a boot process in the HMDD 12, such as when the HMDD 12 turns on, changes from an “idle” state to a ”normal” operating state, changes from a “low-power” operating state to a “normal” operating state, and/or the like.

The HMDD 12 may perform any suitable boot process that initializes and configures the HMDD 12 and its internal components such that the HMDD 12 is operable to provide AR and/or VR services to the user 14. For instance, in some examples, the HMDD 12 may perform an internal self-check to ensure the various components described herein are functioning properly. In some examples, the HMDD 12 may obtain (e.g., from memory 18, server computing system 24, etc.) and load firmware file(s), operating system(s), and/or the like. In some examples, the HMDD 12 may initialize and configure the display device 32. In some examples, the HMDD 12 may establish a connection link with the one or more client computing devices 56. In some examples, the HMDD 12 may load (e.g., from memory 18) and/or synchronize with (e.g., from server computing system 24) the one or more XR applications 44, the one or more XR platforms 46, and/or the like.

In some examples, the HMDD 12 may perform one or more user authentication processes to determine and/or confirm an identify of the user 14. For instance, the HMDD 12 (e.g., via the one or more sensors 34, the one or more cameras 52, etc.) may obtain biometric authentication data 64 associated with the user 14, such as, by way of non-limiting example, eye tracking data, facial recognition data, one or more images of the user 14, and/or the like. The HMDD 12 may determine and, if applicable, confirm an identity of the user 14 based on the biometric authentication data 64. It should be understood that the biometric authentication data 64 may include any data from which the identity of the user 14 may be determined, confirmed, and/or the like.

In some examples, the HMDD 12 may obtain a user profile 66 associated with the user 14. In some examples, the HMDD 12 may obtain the user profile 66 from the memory 18. In some examples, the server computing system 24 may store the user profiles associated with each user serviced by the service provider 26. In such examples, the HMDD 12 may obtain the user profile 66 from the server computing system 24 via the network 22. The user profile 66 may identify and include preferences of the user 14, system parameters 68 associated with the HMDD 12, operational data and preferences associated with one or more mobile applications, and/or the like. For instance, the HMDD 12 may be configured based on the system parameters 68 of the user profile 66.

In some examples, the HMDD 12 may dynamically and intelligently determine and configure a vision setting 70 for the adjustable lenses 58, such as a first vision setting 70-1 for the first adjustable lens 58-1 and a second vision setting 70-2 for the second adjustable lens 58-2. More particularly, the HMDD 12 may determine a visual acuity metric 72 for the user 14. The visual acuity metric 72 may characterize and/or otherwise be associated with a quality and/or a clarity of a vision of the user 14. The HMDD 12 may adjust and/or configure one or more optical characteristics 74 (e.g., refractive index, lens curvature, etc.) of the adjustable lenses 58 (e.g., first optical characteristic 74-1 of the first adjustable lens 58-1, second optical characteristic 74-2 of the second adjustable lens 58-2) based on the visual acuity metric 72 associated with the user 14.

By way of non-limiting example, the HMDD 12 may provide a user-selectable control to the user 14. In some examples, the HMDD 12 may provide the user-selectable control to the user 14 via the display device 32 as an interactive user interface (UI) element (e.g., slider) having a range 76 of different vision settings 78. By way of non-limiting example, the range 76 of different vision settings 78 may include a farsightedness vision setting 78-1 corresponding to a configuration of the adjustable lenses 58 operable to compensate for hyperopia refractive error (e.g., farsightedness), a plurality of intermediate vision settings 78-2 (e.g., at least one of which corresponding to a configuration of the adjustable lenses 58 operable to compensate for no refractive error), and a nearsightedness vision setting 78-3 corresponding to a configuration of the adjustable lenses operable to compensate for myopia refractive error (e.g., nearsightedness). The user 14 may select one of the different vision settings 78 as a selected vision setting via the input device 48, and the HMDD 12 may receive user input data 80 from the input device 48 characterizing the selected vision setting of the range 76 of different vision setting 78. The HMDD 12 may determine the visual acuity metric 72 associated with the user 14 based on the user input data 80 characterizing the selected vision setting.

In some examples, the HMDD 12 may provide a first user-selectable control to the user 14 via the display device 32. The first user-selectable control may include a range of different vision settings for the first adjustable lens 58-1, such as the range 76 described above. The HMDD 12 may also provide a second user-selectable control to the user 14 via the display device 32. The second user-selectable control may include a range of different vision setting for the second adjustable lens 58-2, such as the range 76 described above. The user 14 may individually select a vision setting for each of the first adjustable lens 58-1 and the second adjustable lens 58-2 in a similar manner as described above. The HMDD 12 may receive first user input data 80-1 (e.g., characterizing a selected vision setting for the first adjustable lens 58-1) and second user input data 80-2 (e.g., characterizing a selected vision setting for the second adjustable lens 58-2). The HMDD 12 may determine the visual acuity metric 72 associated with the user 14 based on the first user input data 80-1 and the second user input data 80-2. The HMDD 12 may adjust a first optical characteristic 74-1 (e.g., refractive index, lens curvature, etc.) of the first adjustable lens 58-1 based on the first user input data 80-1 (e.g., characterizing a selected vision setting for the first adjustable lens 58-1); the HMDD 12 may adjust a second optical characteristic 74-2 (e.g., refractive index, lens curvature, etc.) of the second adjustable lens 58-2 based on the second user input data 80-2 (e.g., characterizing a selected vision setting for the second adjustable lens 58-2).

As another non-limiting example, the HMDD 12 (e.g., via the one or more sensors 34) may obtain biometric data 82 associated with the user 14. In some examples, the biometric data 82 may characterize a refractive error of the user 14. For instance, in some examples, the HMDD 12 may include a refractometer 84 in an optical path associated with the user 14 (e.g., having an optical path directed towards the user 14). The refractometer 84 may obtain biometric data 82 characterizing the refractive error of the user 14. In such examples, the HMDD 12 may determine the visual acuity metric 72 associated with the user 14 based on the biometric data 82 obtained by the refractometer 84.

As described herein, the HMDD 12 may adjust and/or configure the one or more optical characteristics 74 (e.g., refractive index, lens curvature, etc.) of the adjustable lenses 58 (e.g., first adjustable lens 74-1 and second adjustable lens 74-2) by applying external stimuli (e.g., electrical stimuli, mechanical stimuli, etc.) to an active medium 60 of the adjustable lenses 58. As an illustrative example, the adjustable lenses 58 may be electro-optical lenses having an active medium 60 that includes liquid crystals. In such examples, the HMDD 12 may provide (e.g., via a controller (not shown), processor device 16, etc.) electrical stimuli to the active medium 60 to adjust the refractive index and/or the lens curvature of the adjustable lenses 58.

As another illustrative example, the adjustable lenses 58 may be opto-mechanical lenses having an active medium 60 that includes a fluid (e.g., fluid silicon, liquid silicone, etc.) within a flexible membrane. In such examples, the HMDD 12 may provide (e.g., via a controller (not shown), processor device 16, etc.) mechanical stimuli to the active medium 60 to adjust the refractive index and/or the lens curvature of the adjustable lenses 58. For instance, in some examples, a controller (e.g., processor device 16) of the HMDD 12 may be coupled to a motor (not shown), which may be operable to provide pressure (e.g., a mechanical stimuli) to the active medium 60 which, in turn, adjusts the refractive index and/or lens curvature of the adjustable lenses 58.

In some examples, the HMDD 12 may fine-tune the vision setting 70 for the adjustable lenses 58. For instance, subsequent to adjusting the optical characteristics 74 of the adjustable lenses 58, the HMDD 12 may receive user feedback data 86. The user feedback data 86 may be provided by the user 14 and received by the HMDD 12 in any suitable manner, such as in a similar manner as described above with reference to the user input data 80. The user feedback data 86 may characterize a tuned vision setting 88 for the adjustable lenses 58. The user 14 may have an increased vision quality and/or vision clarity with the tuned vision setting 88 relative to the vision setting 70. The HMDD 12 may tune (e.g., adjust) the optical characteristic 74 of the adjustable lenses 58 based on the tuned vision setting 88.

Each of the features of the HMDD 12 described above may be operatively connected to one another via a system bus (not shown). Likewise, each of the features of the server computing system 24 described above may be operatively connected to one another via a system bus (not shown). For instance, the system bus (not shown) of the HMDD 12 and/or the system bus (not shown) of the server computing system 24 may be any of several types of bus structures that may further interconnect to a memory bus (with or without a memory controller), a peripheral bus, and/or a local bus using any of a variety of commercially available bus architectures.

The HMDD 12 and server computing system 24 of FIG. 1 and the constituent elements thereof may encompass any one of known digital logic elements, semiconductor circuits, processing cores, and/or memory structures, among other elements, or combinations thereof. Examples described herein are not restricted to any particular arrangement of elements, and it is to be understood that some embodiments of the HMDD 12 may include more or fewer elements than illustrated in FIG. 1. For example, the processor device 16 (of the HMDD 12) and/or the processor device 28 (of the server computing system 24) may further include one or more functional units, instruction caches, unified caches, memory controllers, interconnect buses, and/or additional memory devices, caches, and/or controller circuits, which are omitted from FIG. 1 for the sake of clarity.

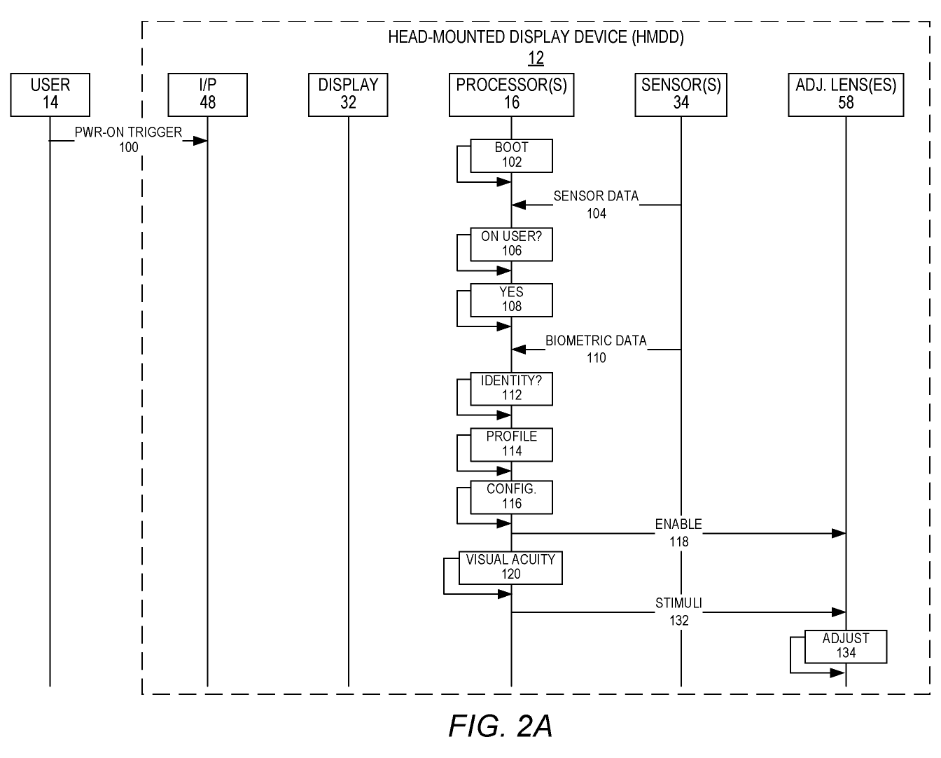

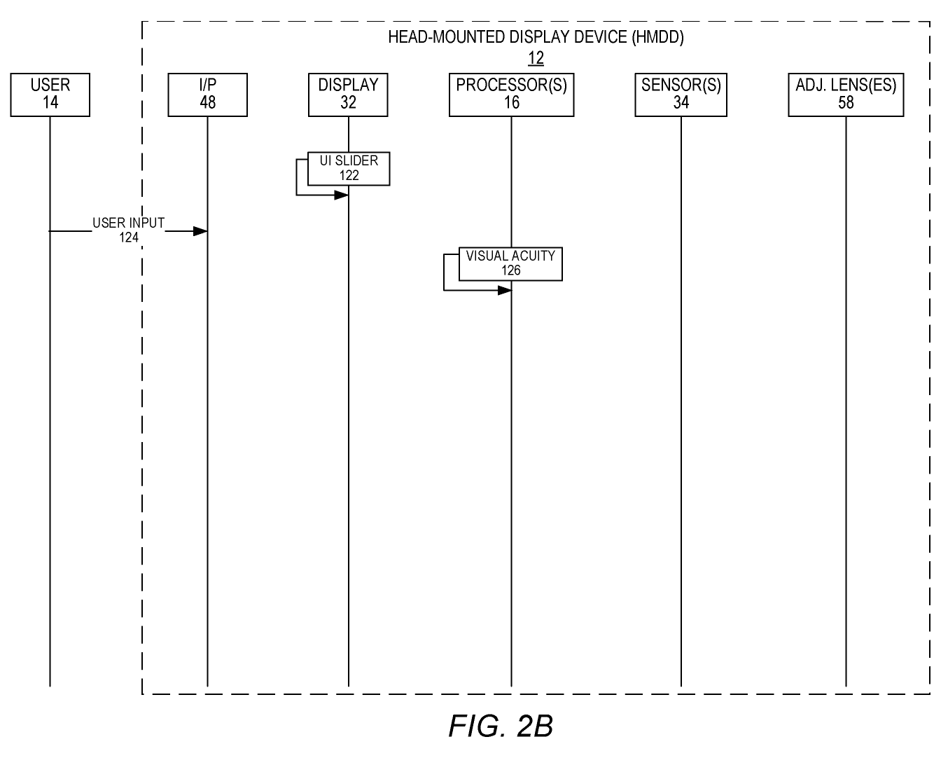

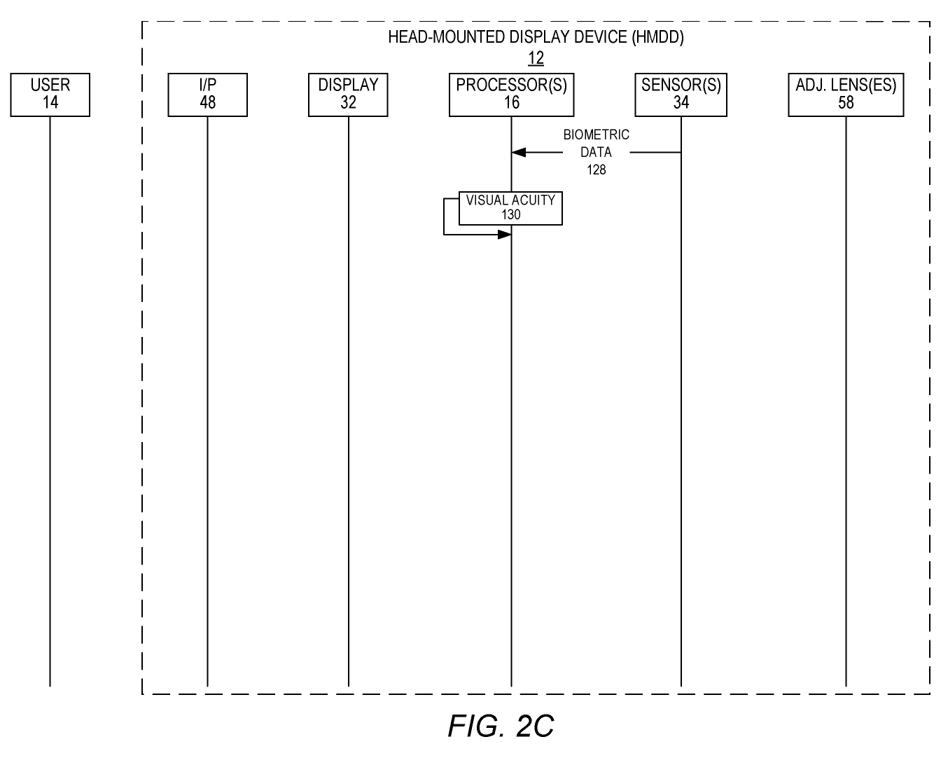

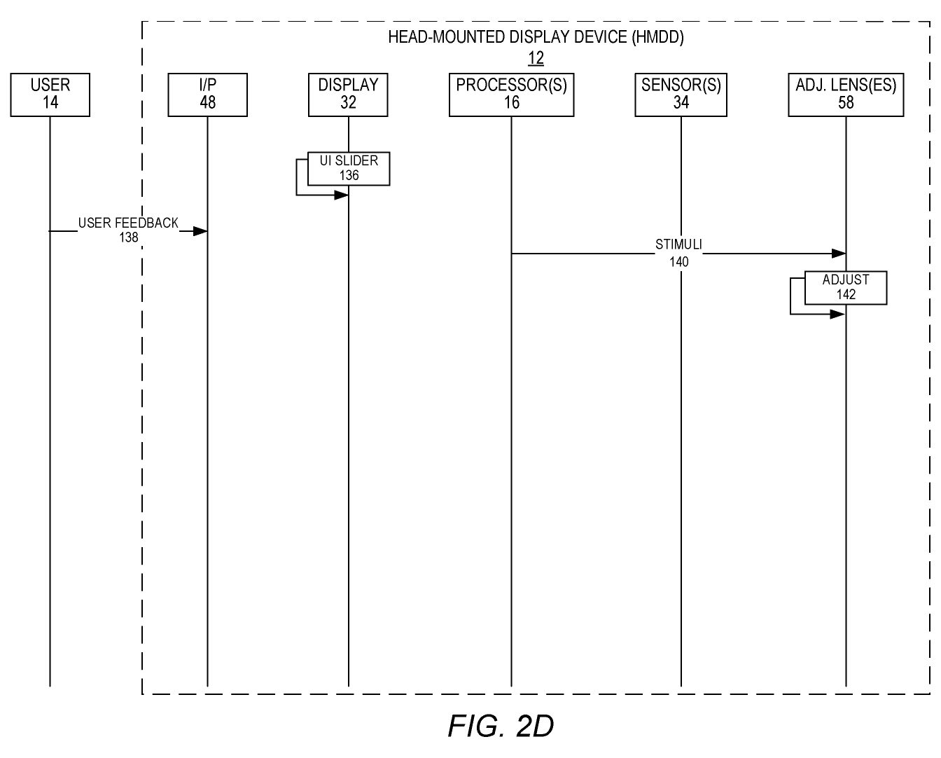

FIGS. 2A-2D are a sequence diagrams illustrating messages communicated between and actions taken by certain components illustrated in FIG. 1 to perform dynamic and adaptive vision correction in a head-mounted display device (HMDD) according to one implementation of the present disclosure. FIGS. 2A-2D will be discussed in conjunction with FIG. 1.

Referring to FIG. 2A, the user 14 provides a power-on trigger to the HMDD 12 by, for example, providing a user in put to the input device 48 (FIG. 2A, step 100). In response, the HMDD 12 (e.g., via processor device 16) initiates a boot process (FIG. 2A, step 102). The HMDD 12 (e.g., processor device 16) obtains sensor data (e.g., biometric data 80) from the sensors 34 (e.g., biometric sensor(s) 38, optical sensor(s) 42) (FIG. 2A, step 104). Based on the sensor data, the HMDD 12 (e.g., processor device 16) determines whether the user 14 is wearing the HMDD 12 (FIG. 2A, step 106).

In response to determining the user 14 is wearing the HMDD 12 (FIG. 2A, step 108), the HMDD 12 (e.g., processor device 16) obtains biometric authentication data 64 associated with the user 14 from the sensors 34 (FIG. 2A, step 110). The HMDD 12 (e.g., processor device 16) determines the identity of the user 14 based on the biometric authentication data 64 (FIG. 2A, step 112). In response to (and confirming) the identity of the user 14, the HMDD 12 (e.g., processor device 16) obtains the user profile 66 associated with the user 14 (FIG. 2A, step 114). In some examples, the user profile 66 may be stored in and retrieved from the memory 18. In some examples, the user profile 66 may be stored in and obtained from the client computing device(s) 56. In some examples, the user profile 66 may be stored in and retrieved from the server computing system 24.

The HMDD 12 (e.g., processor device 16) configures its internal components (e.g., display device(s) 32, sensor(s) 34, XR application(s) 44, XR platform(s) 46, input device(s) 48, output device(s) 50, camera(s) 52, communications interface(s) 54, etc.) based on the system parameters 68 of the user profile 66 (FIG. 2A, step 116). The HMDD 12 (e.g., processor device 16) enables and/or configures the adjustable lenses 58 in a neutral configuration (e.g., operable to compensate for no refractive error in the user 14), such as in one of the plurality of intermediate vision settings 78-2 (FIG. 2A, step 118).

Subsequent to determining the user 14 is wearing the HMDD 12, the HMDD 12 (e.g., processor device 16) determines a visual acuity metric 72 associated with the user 14 (FIG. 2A, step 120). In some examples, the HMDD 12 (e.g., processor device 16) may determine a visual acuity metric 72 for each eye of the user 14 (e.g., visual acuity metric 72-1, visual acuity metric 72-2).

A first non-limiting example and a second non-limiting example of the HMDD 12 determining the visual acuity metric 72 for the user is depicted in FIGS. 2B and 2C, respectively.

As one illustrative example, referring now to FIG. 2B, the HMDD 12 (e.g., via display device 32) provides a user-selectable control (e.g., interactive user interface (UI) slider) to the user 14 that includes the range of different vision settings 78 for the adjustable lenses 58 (FIG. 2B, step 122). The HMDD 12 (e.g., via input device(s) 48) receives user input data 80 associated with the user-selectable control (e.g., associated with a user selection by the user 14) that characterizes a selected vision setting (e.g., vision setting 70) of the range of different vision settings 78 (FIG. 2B, step 124). The HMDD 12 (e.g., processor device 16) determines the visual acuity metric 72 associated with the user 14 based on the user input data 80 (FIG. 2B, step 126).

As another illustrative examples, referring now to FIG. 2C, the HMDD 12 (e.g., via sensors 34) obtains sensor data associated with the user, such as biometric data 82 from the refractometer, that characterizes a refractive error of the user 14 (FIG. 2C, step 128). The HMDD 12 (e.g., processor device 16) determines the visual acuity metric 72 associated with the user 14 based on the sensor data (e.g., biometric data 82) (FIG. 2C, step 130).

Referring again to FIG. 2A, based on the visual acuity metric 72, the HMDD 12 (e.g., processor device 16) provides a stimulus (e.g., electrical stimuli, mechanical stimuli, etc.) to the active medium 60 of the adjustable lenses 58 (FIG. 2A, step 132) to adjust an optical characteristic 74 of the adjustable lenses 58 (e.g., refractive index, lens curvature, etc.) (FIG. 2A, step 134). Subsequently, the HMDD 12 may, in some examples, fine-tune the vision setting 70 (e.g., the optical characteristic 74) of the adjustable lenses 58.

A non-limiting of the HMDD 12 fine-tuning the adjustable lenses 58 is depicted in FIG. 2D.

Referring to FIG. 2D, the HMDD 12 (e.g., via display device 32) provides a user-selectable control (e.g., interactive user interface (UI) slider) to the user 14 that includes the range of different vision settings 78 for the adjustable lenses 58 (FIG. 2D, step 136). The HMDD 12 (e.g., via input device(s) 48) receives user feedback data 86 associated with the user-selectable control (e.g., associated with a user selection by the user 14) that characterizes a tuned vision setting 88 for the adjustable lenses 58 (FIG. 2D, step 138). Based on the tuned vision setting 88, the HMDD 12 (e.g., processor device 16) provides a stimulus (e.g., electrical stimuli, mechanical stimuli, etc.) to the active medium 60 of the adjustable lenses 58 (FIG. 2D, step 140) to tune an optical characteristic 74 of the adjustable lenses 58 (e.g., refractive index, lens curvature, etc.) (FIG. 2D, step 142).

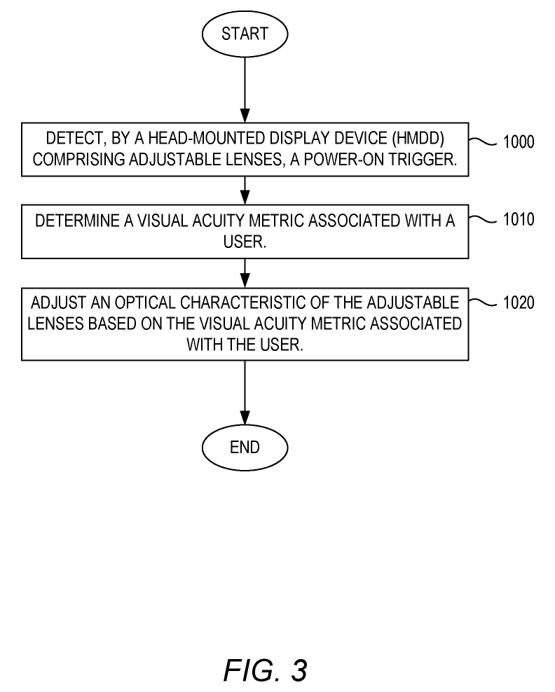

FIG. 3 is a flowchart of an example method for dynamically configuring and/or adjusting the optical characteristics of an HMDD to accommodate the vision needs of users according to one implementation of the present disclosure. FIG. 3 will be discussed in conjunction with FIG. 1. The HMDD 12 detects a power-on trigger, which initiates a boot process of the HMDD 12 (FIG. 3, block 1000). The HMDD 12 determines the visual acuity metric 72 associated with the user 14 of the HMDD 12 (FIG. 3, block 1010). The HMDD 12 adjusts the optical characteristic(s) 74 of the adjustable lenses 58 based on the visual acuity metric 72 associated with the user 14 (FIG. 3, block 1020).

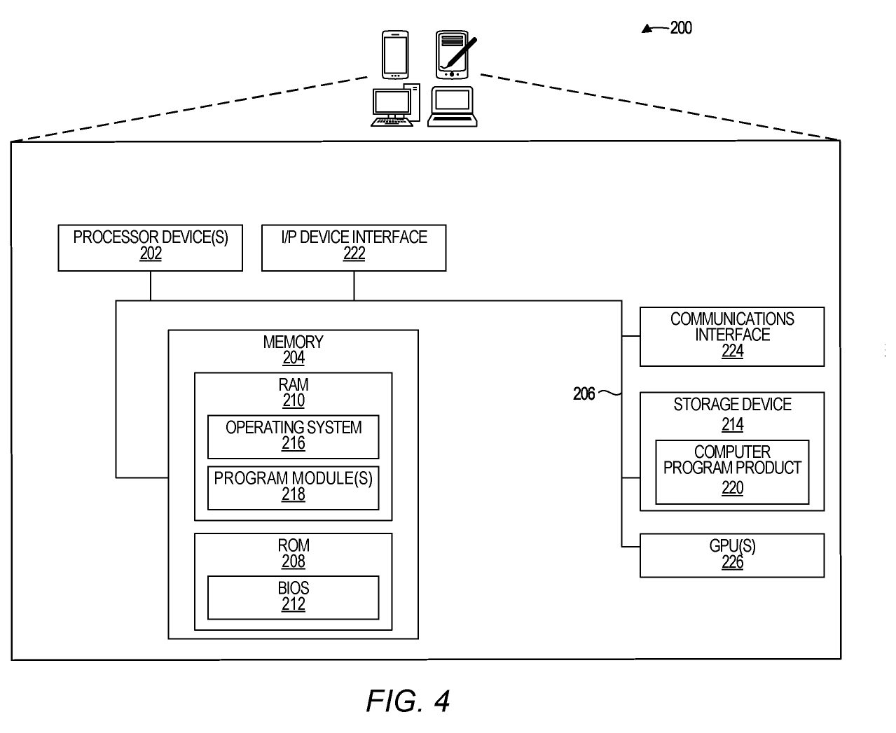

FIG. 4 is a block diagram of a computing device 200, such as the client computing device(s) 56 and/or a computing device of the server computing system 24 of FIG. 1, suitable for implementing examples disclosed herein according to one embodiment.

The computing device 200 may include any computing and/or electronic device capable of including firmware, hardware, and/or executing software instructions to implement the functionality described herein, such as a computer server, computing device, and/or the like. The computing device 200 includes processor device(s) 202 (hereinafter “processor device 202”), a system memory (e.g., memory 204), and a system bus 206. The system bus 206 provides an interface for system components including, but not limited to, the memory 204 and the processor device 202. The processor device(s) 202 may be any commercially available or proprietary processor.

The system bus 206 may be any of several types of bus structures that may further interconnect to a memory bus (with or without a memory controller), a peripheral bus, and/or a local bus using any of a variety of commercially available bus architectures. The memory 204 may include non-volatile memory 208 (e.g., read-only memory (ROM), erasable programmable read-only memory (EPROM), electrically erasable programmable read-only memory (EEPROM), etc.), and volatile memory 210 (e.g., random-access memory (RAM)). A basic input/output system (BIOS) 212 may be stored in the non-volatile memory 208 and may include the basic routines that help to transfer information between elements within the computing device 200. The volatile memory 210 may also include a high-speed RAM, such as static RAM, for caching data.

The computing device 200 may further include or be coupled to a non-transitory computer-readable storage medium, such as a storage device 214, which may comprise, for example, an internal or external hard disk drive (HDD) (e.g., enhanced integrated drive electronics (EIDE) or serial advanced technology attachment (SATA)), HDD (e.g., EIDE or SATA) for storage, flash memory, or the like. The storage device 214 and other drives associated with computer-readable media and computer-usable media may provide non-volatile storage of data, data structures, computer-executable instructions, and the like.

A number of modules can be stored in the storage device 214 and in the volatile memory 210, including an operating system 216 and one or more program modules 218, which may implement the functionality described herein in whole or in part. All or a portion of the examples may be implemented as a computer program product 220 stored on a transitory or non-transitory computer-usable or computer-readable storage medium, such as the storage device 214, which includes complex programming instructions, such as complex computer-readable program code, to cause the processor device 202 to carry out the steps described herein. Thus, the computer-readable program code may comprise software instructions for implementing the functionality of the examples described herein when executed on the processor device 202. The processor device 202 may serve as a controller and/or or a control system for the computing device 200 that is to implement the functionality described herein.

An operator (e.g., user 14) may also be able to enter one or more configuration commands through a keyboard (not illustrated), a pointing device such as a mouse (not illustrated), or a touch-sensitive surface such as a display device (not illustrated). Such input devices may be connected to the processor device 202 through an input device interface 222 coupled to the system bus 206 but can be connected through other interfaces such as a parallel port, an Institute of Electrical and Electronic Engineers (IEEE) 1394 serial port, a Universal Serial Bus (USB) port, an IR interface, and the like.

The computing device 200 may also include a communications interface 224 suitable for communicating with the network 22 of FIG. 1 as appropriate or desired. The computing device 200 includes one or more GPUs 226.

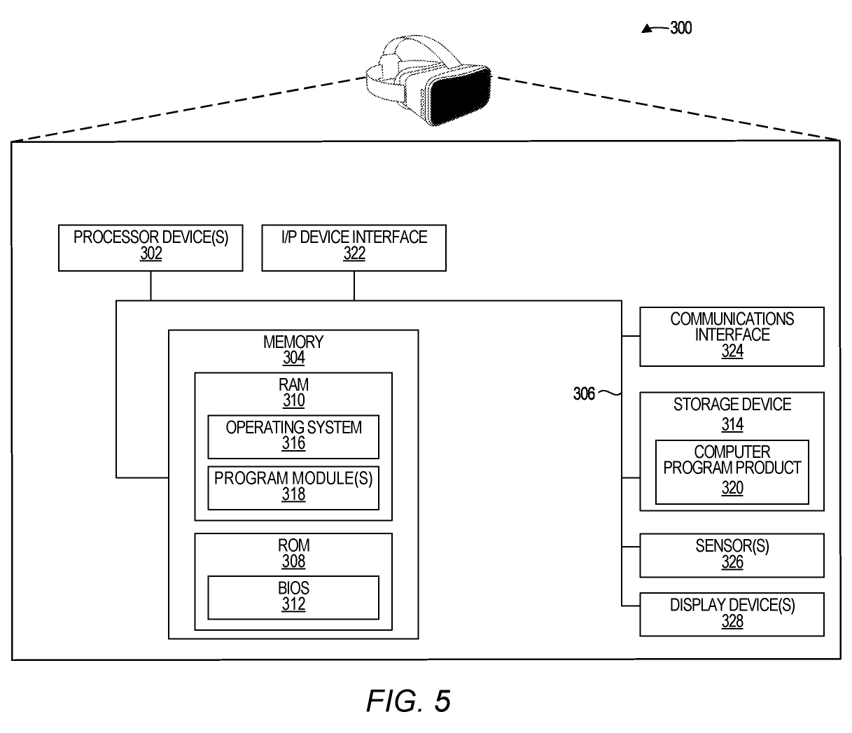

FIG. 5 is a block diagram of a head-mounted display device (HMDD) 300, such as the HMDD 12 of FIG. 1, suitable for implementing the examples disclosed herein according to one embodiment.

The HMDD 300 may include any computing and/or electronic device capable of including firmware, hardware, and/or executing software instructions to implement the functionality described herein. The HMDD 300 includes processor device(s) 302 (hereinafter “processor device 302”), a system memory (e.g., memory 304), and a system bus 306. The system bus 306 provides an interface for system components including, but not limited to, the memory 304 and the processor device 302. The processor device(s) 302 may be any commercially available or proprietary processor.

The system bus 306 may be any of several types of bus structures that may further interconnect to a memory bus (with or without a memory controller), a peripheral bus, and/or a local bus using any of a variety of commercially available bus architectures. The memory 304 may include non-volatile memory 308 (e.g., read-only memory (ROM), erasable programmable read-only memory (EPROM), electrically erasable programmable read-only memory (EEPROM), etc.), and volatile memory 310 (e.g., random-access memory (RAM)). A basic input/output system (BIOS) 312 may be stored in the non-volatile memory 308 and may include the basic routines that help to transfer information between elements within the HMDD 300. The volatile memory 310 may also include a high-speed RAM, such as static RAM, for caching data.

The HMDD 300 may further include or be coupled to a non-transitory computer-readable storage medium, such as a storage device 314, which may comprise, for example, an internal or external hard disk drive (HDD) (e.g., enhanced integrated drive electronics (EIDE) or serial advanced technology attachment (SATA)), HDD (e.g., EIDE or SATA) for storage, flash memory, or the like. The storage device 314 and other drives associated with computer-readable media and computer-usable media may provide non-volatile storage of data, data structures, computer-executable instructions, and the like.

A number of modules can be stored in the storage device 314 and in the volatile memory 310, including an operating system 316 and one or more program modules 318, which may implement the functionality described herein in whole or in part. All or a portion of the examples may be implemented as a computer program product 320 stored on a transitory or non-transitory computer-usable or computer-readable storage medium, such as the storage device 314, which includes complex programming instructions, such as complex computer-readable program code, to cause the processor device 302 to carry out the steps described herein. Thus, the computer-readable program code may comprise software instructions for implementing the functionality of the examples described herein when executed on the processor device 302. The processor device 302 may serve as a controller and/or or a control system for the HMDD 300 that is to implement the functionality described herein.

An operator (e.g., user 14) may also be able to enter one or more configuration commands through buttons and/or other input controls integrated into the HMDD 300 (e.g., input device(s) 48), via an external interface such as a keyboard (not illustrated), a pointing device such as a mouse (not illustrated), a wireless VR controller (not illustrated). The operator (e.g., user 14) may also be able to enter one or more configuration commands through interactions with objects within a virtual environment, such as through hand gestures, finger gestures, and/or the like. Such input devices may be connected to the processor device 302 through an input device interface 322 coupled to the system bus 306 but can be connected through other interfaces such as a parallel port, an Institute of Electrical and Electronic Engineers (IEEE) 1394 serial port, a Universal Serial Bus (USB) port, an IR interface, and the like. In some examples, the operator may provide input through hand motions that are tracked by cameras (not illustrated) provided by the HMDD 300 and/or present in physical proximity to the operator.

The HMDD 300 may also include a communications interface 324 suitable for communicating with the network 22 of Figure 1 as appropriate or desired. The HMDD 300 includes one or more sensor(s) 326 (e.g., IMU(s) 36, biometric sensor(s) 38, depth sensor(s) 40, optical sensor(s) 42, refractometer(s) 84, etc.), a display device 328 (e.g., display device(s) 32), and adjustable lenses 330 (e.g., adjustable lenses 58). In some examples, the HMDD 300 does not include a GPU.

Individuals will recognize improvements and modifications to the preferred examples of the disclosure. All such improvements and modifications are considered within the scope of the concepts disclosed herein and the claims that follow.

What is claimed is:

1. A method, comprising:
determining, by a head-mounted display device (HMDD) comprising adjustable lenses, a visual acuity metric associated with a user of the HMDD; and
adjusting, by the HMDD, an optical characteristic of the adjustable lenses based on the visual acuity metric associated with the user.

2. The method of claim 1, wherein determining the visual acuity metric associated with the user comprises:
providing, by a display of the HMDD, a user-selectable control to the user, the user-selectable control comprising a range of different vision settings for the adjustable lenses;
receiving, by the HMDD, user input data associated with the user-selectable control, the user input data characterizing a selected vision setting of the range of different vision settings; and
determining, by the HMDD, the visual acuity metric associated with the user based on the user input data.

3. The method of claim 2, wherein the user-selectable control is an interactive user interface (UI) slider comprising the range of different vision settings, the range of different vision settings comprising:
a farsightedness vision setting, the farsightedness vision setting corresponding to a configuration of the adjustable lenses operable to compensate for hyperopia refractive error;
a plurality of intermediate vision settings, at least one of the plurality of intermediate corresponding to a neutral configuration of the adjustable lenses operable to compensate for no refractive error; and
a nearsightedness vision setting, the nearsightedness vision setting corresponding to a configuration of the adjustable lenses operable to compensate for myopia refractive error.

4. The method of claim 2, wherein the adjustable lenses comprise a first lens and a second lens, and wherein providing the user-selectable control to the user comprises:
providing, by the display of the HMDD, a first user-selectable control, the first user-selectable control comprising a range of different vision settings for the first lens; and
providing, by the display of the HMDD, a second user-selectable control that is different from the first user-selectable control, the second user-selectable control comprising a range of different vision settings for the second lens.

5. The method of claim 4, wherein:
receiving the user input data comprises:
receiving, by the HMDD, first user input data associated with the first user-selectable control, the first user input data characterizing a selected vision setting for the first lens; and
receiving, by the HMDD, second user input data associated with the second user-selectable control, the second user input data characterizing a selected vision setting for the second lens; and
determining the visual acuity metric associated with the user comprises:
determining, by the HMDD, the visual acuity metric associated with the user based on the first user input data and the second user input data.

6. The method of claim 5, wherein adjusting the optical characteristic of the adjustable lenses comprises:
adjusting, by the HMDD, one of a refractive index or a lens curvature of the first lens based on the first user input data characterizing the selected vision setting for the first lens; and
adjusting, by the HMDD, one of a refractive index or a lens curvature of the second lens based on the second user input data characterizing the selected vision setting for the second lens.

7. The method of claim 1, wherein determining the visual acuity metric associated with the user comprises:
obtaining, by a sensor of the HMDD, biometric data associated with the user, the biometric data characterizing a refractive error of the user; and
determining, by the HMDD, the visual acuity metric associated with the user based on the biometric data.

8. The method of claim 7, wherein the sensor is a refractometer.

9. The method of claim 7, further comprising:
subsequent to adjusting the optical characteristic of the adjustable lenses based on the visual acuity metric, receiving, by the HMDD, user feedback data characterizing a tuned vision setting for the adjustable lenses; and
tuning, by the HMDD, the optical characteristic of the adjustable lenses based on the tuned vision setting.

10. The method of claim 1, wherein the adjustable lenses are electro-optical lenses, and wherein adjusting the optical characteristic of the adjustable lenses comprises:
providing, by the HMDD based on the visual acuity metric associated with the user, electrical stimuli to an active medium of the electro-optical lenses to adjust a refractive index of the electro-optical lenses, the active medium comprising liquid crystals.

11. The method of claim 1, wherein the adjustable lenses are opto-mechanical lenses, and wherein adjusting the optical characteristic of the adjustable lenses comprises:
providing, by the HMDD based on the visual acuity metric associated with the user, mechanical stimuli to an active medium of the opto-mechanical lenses to adjust a lens curvature of the opto-mechanical lenses, the active medium comprising a fluid within a flexible membrane.

12. The method of claim 11, wherein the fluid is liquid silicone.

13. The method of claim 1, wherein, prior to determining the visual acuity metric associated with the user, the method comprises:
detecting, by the HMDD, a power-on trigger, the power-on trigger initiating a boot process of the HMDD;
in response to detecting the power-on trigger, determining, by the HMDD, that the user is wearing the HMDD;
in response to determining that the user is wearing the HMDD, obtaining, by the HMDD, biometric authentication data associated with the user; and
determining, by the HMDD, an identity of the user based on the biometric authentication data.

14. The method of claim 13, further comprising:
in response to determining the identity of the user, obtaining, by the HMDD, a user profile associated with the user; and
configuring, by the HMDD, system parameters of the HMDD based on the user profile.

15. A head-mounted display device (HMDD), comprising:
a display;
adjustable lenses proximate the display, the adjustable lenses comprising an active medium;
a memory; and
a processor device coupled to the memory and operable to:
determine a visual acuity metric associated with a user of the HMDD; and
adjust an optical characteristic of the adjustable lenses based on the visual acuity metric associated with the user.

16. The HMDD of claim 15, wherein, to determine the visual acuity metric associated with the user, the processor device is operable to:
provide, by the display, a user-selectable control to the user, the user-selectable control comprising a range of different vision settings for the adjustable lenses;
receive user input data associated with the user-selectable control, the user input data characterizing a selected vision setting of the range of different vision settings; and
determine the visual acuity metric associated with the user based on the user input data.

17. The HMDD of claim 15, further comprising:
a sensor having an optical path directed towards the user,
wherein, to determine the visual acuity metric associated with the user, the processor device is operable to:
obtain, by the sensor, biometric data associated with the user, the biometric data characterizing a refractive error of the user; and
determine the visual acuity metric associated with the user based on the biometric data.

18. The HMDD of claim 17, wherein the sensor is a refractometer.

19. The HMDD of claim 15, wherein the adjustable lenses are one of:
electro-optical lenses comprising an active liquid crystal medium, the active liquid crystal medium operable to adjust a refractive index of the electro-optical lenses in response to applied electrical stimuli; or
opto-mechanical lenses comprising an active fluid medium within a flexible membrane, the active fluid medium operable to adjust a lens curvature of the opto-mechanical lenses in response to applied mechanical stimuli.

20. A non-transitory computer-readable medium that includes executable instructions configured to cause a processor device to:
determine a visual acuity metric associated with a user of a head-mounted display device (HMDD) comprising adjustable lenses; and
adjust an optical characteristic of the adjustable lenses based on the visual acuity metric associated with the user.
